(19) United States
(12) Patent Application Publication
(10) Pub. No.: US 2026/0240926 A1
(43) Pub. Date: Aug. 20, 2026

(54) METHOD OF MAKING A PROBIOTIC FERMENTED PRODUCT

(71) Applicant: UNIVERSITY OF SOUTH FLORIDA, Tampa, FL (US)

(72) Inventors: Brandi C. MILLER, Tampa, FL (US); Hariom YADAV, Tampa, FL (US); Shalini JAIN, Land O Lakes, FL (US)

(21) Appl. No.: 19/158,955

(22) PCT Filed: Feb. 27, 2024
(86) PCT No.: PCT/US2024/017507

§ 371 Date: Aug. 22, 2025

Related U.S. Application Data
(60) Provisional application No. 63/487,561, filed on Feb. 28, 2023.
(60) Provisional application No. 63/606,900, filed on Dec. 6, 2023.

(51) Int. Cl. A61K 35/747; A61K 35/20; A61K 35/745; A61P 1/14; C12N 1/20; C12P 7/42; C12R 1/225

(52) U.S. Cl. CPC A61K 35/747; A61K 35/20; A61K 35/745; A61P 1/14; C12N 1/20; C12P 7/42; C12R 2001/225

(57) ABSTRACT

A probiotic fermented product is disclosed comprising at least four live strains of bacteria, wherein said bacteria are: Lactobacillus reuteri, Lactobacillus rhamnosus, Lactobacillus paracasei and Bifidobacterium longum that produce 3-hydroxybutyrate in the probiotic fermented product. Also disclosed herein is a method of treating dysbiosis in a subject with the probiotic fermented product, wherein the dysbiosis can be aging-associated.

### CROSS-REFERENCE TO RELATED APPLICATIONS

This application claims benefit of U.S. Provisional Application No. 63/487,561, filed Feb. 28, 2023, and U.S. Provisional Application No. 63/606,900, filed Dec. 6, 2023, both of which are hereby incorporated herein by reference in their entirety.

### BACKGROUND

Probiotics are live microorganisms that are naturally abundant in the gut. Common probiotics include species of Lactobacillus, Bifidobacterium, and Streptococcus. Probiotics impact health by modulating the gut microbiome and its metabolites, in a strain-specific manner. Many studies have elucidated the beneficial effects of probiotics on metabolism, but their effects on aging and aging-induced dysbiosis remain elusive.

Thus, there is a need to address the aforementioned problems and other shortcomings associated by developing a composition or method to treat aging-induced dysbiosis.

### SUMMARY

In one aspect, the present invention discloses a probiotic fermented product comprising at least four live strains of bacteria, wherein said bacteria are: Lactobacillus reuteri, Lactobacillus rhamnosus, Lactobacillus paracasei and Bifidobacterium longum subsp. longum.

In another aspect, the present invention discloses a method of producing 3 hydroxybutyrate, wherein the method comprises 1) providing a fermentable food product and 2) fermenting the food product by providing the combination of at least four of the live strains of bacteria, wherein the bacteria comprise Lactobacillus reuteri, Lactobacillus rhamnosus, Lactobacillus Paracasei and Bifidobacterium longum subsp. longum, and the at least four live strains of bacteria which produce 3-hydroxybutyrate.

In another aspect, the present invention discloses a method of treating aging-associated-, cancer-treatment associated-, medicine-associated, antibiotics associated, metabolic syndrome including diabetes, obesity, cardiovascular disorders, kidney and liver disorders, muscle and brain disorders, inflammatory bowel diseases, inflammatory bowel syndrome-associated dysbiosis in a subject, using the probiotic fermented product wherein the subject is treated with a therapeutically effective dose of the probiotic fermented product. In some embodiments, the aging-associated dysbiosis is induced by chemotherapy. In some embodiments, the probiotics are in a therapeutically effective dose. In some embodiments, the subject is a human. In some embodiments, the subject is administered the novel probiotic fermented product at least once a day or multiple times.

In some embodiments, the strain of L reuteri is HL 278. In some embodiments, the strain of L. rhamnosus is HL 279. In some embodiments, the strain of L. paracasei is HL 260. In some embodiments, the strain of B. longum subsp. longum is HL 82. In one aspect, the novel probiotic fermented product is low-, medium-and normal-fat milk from cow, goat, camel, or buffalo. In some embodiments, the product to be fermented is a vegan or plant-based option selected from but not limited to soy-milk, almond milk, cashew milk, oat milk, coconut milk, rice milk or other milk.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 (A-B) shows a flow chart indicating the process of strain selection in FIG. 1A and the fermented probiotic milk in FIG. 1B.

FIG. 2 shows the methodology for the treatment of C. elegans with live bacteria and measurement of their lifespan.

FIG. 3 shows that live bacteria demonstrate anti-aging effects in C. elegans.

FIG. 4 shows the preparation of fermented milk supernatants/extracts for the treatment of C. elegans.

FIG. 5 shows that fermented supernatants (5% [v/v]) demonstrate anti-aging effects in C. elegans.

FIG. 6 (A-B) shows that live bacteria coagulate low-fat milk strain-specifically as in FIG. 6A and cause change in pH as shown in FIG. 6B.

FIG. 7 (A-B) shows that (FIG. 7A) 1% (v/v) and (FIG. 7B) 5% (v/v) fermented milk bacteria demonstrate anti-aging effects in C. elegans shown by the percent viability of C. elegans.

FIG. 8 (A-C) shows a selection of four strains of bacteria in FIG. 8A and the 6-week storage stability of fermented probiotic development in terms of pH as in FIG. 8B and number of colony forming units (CFU)/ml as in FIG. 8C.

FIG. 9 (A-B) shows that fermented bacteria produce unique polar metabolites (FIG. 9A) and lipids (FIG. 9B).

FIG. 10 (A-C) shows the preparation of fermented milk. FIG. 10A shows a flowchart of the process for assessing coagulation in low-fat milk, determining the growth (coagulation) of bacteria in low-fat milk, and sensory evaluations based on published criteria. FIG. 10B shows assessment of percent change in pH of the PFM caused by each bacterial strain. FIG. 10C shows the assessment of percent syneresis (the percentage of liquid remaining out of total starting volume) for each strain.

FIG. 11 shows optimization of the growth of four selected strains in media and 16-hour growth curves in MRS media.

FIG. 12 (A-D) shows the OD600 vs. CFU/mL standard curves for strains (FIG. 12A) HL-278, (FIG. 12B) HL-279, (FIG. 12C) HL-260 and (FIG. 12D) HL-82. OD600 and bacterial colonies were quantified every 2 hours for up to 16 hours to generate standard curves.

FIG. 13 (A-B) shows optimal growth time of four strains together in low-fat milk. FIG. 13A shows a flowchart of the process of optimization of the fermentation of the strains together in low-fat animal milk. FIG. 13B shows images of the fermentation of the strains together in low-fat animal milk, starting at 8 H and continuing to 16 H. This shows the coagulation of these strains together over time.

FIG. 14 (A-D) shows optimization of the growth of four selected strains in low-fat animal milk following a 16-hour fermentation. FIG. 14A shows the comparison of percent change in pH caused by different strains and the multi-strain combination. FIG. 14B shows comparison of the CFU/ml resulting from fermentation of the strains alone and in combination (multi-strain). FIG. 14C shows the comparison of pH of low-fat cow milk versus PFM. FIG. 14D shows comparison of the CFU/ml in milk versus PFM.

FIG. 15 (A-B) shows the establishment of a mouse model for testing PFM. FIG. 15A shows the treatment groups of mice. FIG. 15B shows timeline of treatment and testing.

FIG. 16 (A-E) shows the establishment of a model of chemotherapy induced dysbiosis in C57BL/6 mice using 5-Fluorouracil (5-FU) chemotherapy. 5-FU accelerated body weight loss (FIG. 16A), reduced microbiome diversity (FIG. 16B), increased gut permeability (FIG. 16C), damaged intestinal villi in the ileum (FIG. 16D), and impaired cognitive function (FIG. 16E).

FIG. 17 (A-G) shows that PFM protects from 5-Fluorouracil-induced abnormalities in mice. FIG. 17A shows the groups of mice and interventions used. FIG. 17B shows timeline of treatment and testing. PFM protected from 5-FU-induced effects, including accelerated body weight loss (FIG. 17C), reduced microbiome diversity (FIG. 17D), elevated gut permeability (FIG. 17E), damaged intestinal villi in the ileum (FIG. 17F), and cognitive impairment (FIG. 17G).

FIG. 18 (A-F) shows that PFM protects from 5-FU-induced changes in gut and brain physiology, compared to non-fermented probiotics (NFP). FIG. 18A shows the groups of mice and interventions used. FIG. 18B shows timeline of treatment and testing. PFM protected from accelerated body weight loss (FIG. 18C), reduced microbiome diversity (FIG. 18D), increased gut permeability (FIG. 18E), and impaired cognitive function (FIG. 18F), compared to NFP.

FIG. 19 shows that fermentation by the four strains of bacteria produces unique metabolites. 3-hydroxybutyrate (also called β-hydroxybutyrate, BHB) is a metabolite produced by the four live strains of bacteria that can be used to mediate the protective effects of PFM.

FIG. 20 shows the establishment of an in vitro cell culture model for measuring intestinal barrier permeability. This in vitro model can be used to understand how BHB (identified in FIG. 19) impacts gut permeability in an animal model.

FIG. 21 (A-B) shows transepithelial electrical resistance (TEER) after (FIG. 21A) and change in TEER during 4-hour 5-FU treatment (FIG. 21B).

FIG. 22 (A-D) shows optimization of growth of four strains, (FIG. 22A) Lactobacillus reuteri, (FIG. 22B) Lactobacillus rhamnosus, (FIG. 22C) Lactobacillus paracasei and (FIG. 22D) Bifidobacterium longum subsp. longum, in multiple plant-based medias, compared to standard media (MRS). Tryptone MRS was selected for further experiments.

FIG. 23 (A-D) shows OD600 vs. CFU/ml standard curves of four strains, (FIG. 23A) HL-278, (FIG. 23B) HL-279, (FIG. 23C) HL-260 and (FIG. 23D) HL-82, in plant-based media.

FIG. 24 (A-D) shows optimization of four strains in almond milk. FIG. 24A shows the percent change in pH of almond milk after 16 hours of fermentation for the single strains and strains in combination. FIG. 24B shows CFU/ml of the 4 strains of bacteria and multi-strain formulation after 16 hours of fermentation of almond milk. FIG. 24C shows a comparison of change in pH in non-fermented and probiotic fermented almond milk. FIG. 24D shows a comparison of CFU/ml in non-fermented and probiotic fermented almond milk.

FIG. 25 shows the timeline for a 6-week storage study.

FIG. 26 (A-D) shows six-week storage stability of PFM. FIG. 26A shows a comparison of pH stability in PFM prepared in low-fat milk and almond milk. FIG. 26B shows a comparison of percent change in pH in PFM prepared in low-fat milk and almond milk. FIG. 26C shows a comparison of CFU/ml in PFM prepared in low-fat milk and almond milk. FIG. 26D shows a comparison of percent change of CFU/ml in PFM prepared in low-fat milk and almond milk.

FIG. 27 (A-E) shows sensory evaluations of aroma in FIG. 27A, appearance in FIG. 27B, flavor in FIG. 27C, mouth feel in FIG. 27D and aftertaste in FIG. 27E, of PFM-low fat animal milk, prepared with various concentrations of pectin (used to improve or optimize texture).

FIG. 28 (A-E) shows sensory evaluations of aroma in FIG. 28A, appearance in FIG. 28B, flavor in FIG. 28C, mouth feel in FIG. 28D and aftertaste in FIG. 28E, of PFM-almond milk, prepared with various concentrations of pectin (used to improve or optimize texture).

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

In this specification and in the claims which follow, reference will be made to a number of terms which shall be defined to have the following meanings:

As used herein, the singular forms “a,” “an” and “the” include plural referents unless the context clearly dictates otherwise. Thus, for example, reference to a “metal” includes examples having two or more such “metals” unless the context clearly indicates otherwise.

Ranges can be expressed herein as from “about” one particular value, and/or to “about” another particular value. When such a range is expressed, another example includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “subject” is defined herein to include animals such as mammals, including, but not limited to, primates (e.g., humans), cows, sheep, goats, horses, pigs, dogs, cats, rabbits, rats, mice, birds like poultry and the like. In some embodiments, the subject is a human.

The term “therapeutically effective” refers to the amount of the composition used is of sufficient quantity to ameliorate one or more causes or symptoms of a disease or disorder. Such amelioration only requires a reduction or alteration, not necessarily elimination. The terms “therapeutically effective amount” or “therapeutically effective dose” also refers to the amount of a compound such as probiotic fermented milk that will elicit the biological or medical response of a tissue, system, animal, or human. In some embodiments, a desired response is decrease in dysbiosis or aging-induced dysbiosis. In some instances, a desired biological or medical response is achieved following administration of multiple dosages of the composition to the subject over a period of days, weeks, or years.

The term “syneresis” refers to the contraction of a material for example, a gel, accompanied by the separating out of liquid.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “treat”, “treating”, “treatment”, and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the invention may be applied preventively, prophylactically, palliatively, or remedially. Treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

In some instances, the terms “treat,” “treating”, “treatment” and grammatical variations thereof, include partially or completely reducing the aging-induced dysbiosis, and reducing the severity of aging-induced dysbiosis as compared with prior to treatment of the subject or as compared with the incidence of such symptom in a general or study population. The terms “treat,” “treating”, “treatment” and grammatical variations thereof, can also include partially or completely decreasing the aging-induced dysbiosis as compared with prior to treatment of the subject or as compared with the incidence of such symptom in a general or study population.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

The term “microbiota” refers to the range of microorganisms that may be commensal, symbiotic, or pathogenic found in and on all multicellular organisms, including plants and animals. These include bacteria, archaea, protists, fungi, and viruses and have been found to be crucial for immunologic, hormonal, and metabolic homeostasis of the host.

Used herein, the term “probiotics” refers to live microorganisms promoted with claims that they provide health benefits when consumed, generally by improving or restoring the gut flora.

Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunomodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, cdatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120(VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, iscodermolide, caminomycin, aminopterin, and hexamethyl melamine.

### General Description

It is understood that the invention of the present disclosure can be used in combination with the various compositions, methods, products, and applications disclosed herein.

### Probiotic Fermented Product

Disclosed herein is a probiotic fermented product. A probiotic refers to live microorganisms that provide health benefits when consumed, generally by improving or restoring the gut flora. As disclosed herein, fermentation is a process involving the chemical breakdown of a substance or product by bacteria or fungi to produce several compounds. In some embodiments, fermentation results in production of organic acids (lactic acid, acetic acid, formic acid, phenylacetic acid, and propionic acid), alcohol, carbon dioxide, reuterin, diacetyl, hydrogen peroxide, bacteriocins, and peptides. As disclosed herein, fermentation is one of the oldest methods of food preservation comprising inhibition of the growth of spoilage bacteria. In some embodiments it is a technique to reduce or eliminate the presence of toxic compounds and to inhibit pathogenic bacteria present in the food product. In some embodiments, fermentation is associated with water reduction due to the addition of a sufficiently large salt quantity in order to extend the shelf life of fruits, vegetables, meat, and fish. As shown herein, the most common fermented foods that naturally contain probiotics, or have probiotics added to them, include dairy products like yogurt, cultured buttermilk, sour cream, kefir, koumiss, ymer, filmjölk and others like kombucha, sauerkraut, pickles, miso, tempeh, kimchi, gochujang, wine, ergo, garris, gundruk, rusip, brem, sourdough bread and some cheeses,

In some embodiments, lactic acid-producing bacteria (LAB) are found naturally in the human gastrointestinal tract and other parts of the body. As disclosed herein, the LAB isolated from fermented foods have the ability to aid in digestive health and are known as probiotics. In some embodiments, the probiotics can replace antibiotics in overcoming pathogenic bacteria. In some embodiments, the probiotics possess the ability to improve the immune system and strengthen the body against pathogenic bacteria. Thus, as disclosed herein, consuming probiotics is useful for maintaining health against pathogenic bacteria in the gut microbiota, and maintaining the normal balance of gut microbiota helps to improve digestive health as well as the immune system. In some embodiments, probiotics improve various health conditions such as, for example, irritable bowel syndrome, diarrhea, high cholesterol, yeast infections, bacterial vaginosis and dysbiosis, wherein dysbiosis refers to a change in the environment associated with a pathological state.

As disclosed herein, age-or disease-related changes in composition of the gut microbiome or dysbiosis can lead to a disruption in gut homeostasis in terms of integrity and overall physiology wherein, gut dysbiosis leads to chronic low-grade inflammation, obesity, diabetes, cardiovascular disease, behavioral disorders, and neurodegenerative disease.

Also disclosed herein, the product to be fermented can be fat free, low-fat milk, 1%, 2%, whole milk, lactose-free or other vegan or plant-based option selected from but not limited to soy-milk, almond milk, cashew milk, rice milk, oat milk, hemp milk, coconut milk, goat milk, buffalo milk, sheep milk or camel milk. As disclosed herein, the probiotic fermented product is administered orally to the subject. In some embodiments, the probiotics fermented product can be formulated as a medicament. In some embodiments the medicament is administered as a liquid, semi-solid, capsule, tablet, pill, wafer, powder, frozen or freeze-dried. In some embodiments the medicament may include pharmaceutically acceptable ingredients including pharmaceutically acceptable carriers, adjuvants, buffers, preservatives, stabilizers, sweetening agents, other flavoring reagent, or coloring agents.

As disclosed herein, 36 strains of bacteria were screened during the development of the probiotic fermented product. In some embodiments the strains of bacteria are D3.5, D4.4, D6.1, D6.2, D7.5, D10.4, D13.4, HL-34, HL-64, HL-73, HL-82, HL-258, HL-259 HL-260, HL-261, HL-262, HL-263, HL-264, HL-265, HL-266, HL-267, HL-268, HL-269, HL-270, HL-271 HL-272 HL,273, HL-274, HL-276, HL-278 HL-279 HL-280, HL-281, HL-282, HL-283, HL-285. In some embodiments the bacteria are of human-origin. As disclosed herein, the 36 strains of bacteria are screened for effects on the lifespan of Caenorhabditis elegans. In some embodiments, 27 strains that increased the lifespan of the C. elegans. In further embodiments, the 27 strains were screened for fermenting bacteria. As disclosed herein, 7 strains out of the 27 were selected as the fermenting bacteria.

As disclosed herein, the product to be fermented is fermented by at least four live strains of bacteria, wherein said bacteria are: Lactobacillus reuteri, Lactobacillus rhamnosus, Lactobacillus paracasei and Bifidobacterium longum subsp. longum. In some embodiments, the strain of L reuteri is HL-278. In some embodiments, the strain of L. rhamnosus is HL-279. In some embodiments, the strain of L. paracasei is HL-260. In some embodiments, the strain of B. longum is HL-82. In some embodiments, the strain of L. rhamnosus is HL-282. In some embodiments, the strain of L. rhamnosus is HL 284. In some embodiments, the strain of L. rhamnosus is HL-285. In some embodiments, the bacterial strains can be engineered.

Method of Producing 3-hydroxybutyrate

Also disclosed here is a method of producing 3-hydroxybutyrate, wherein the method comprises: providing a fermentable food product; and further fermenting the food product by providing a combination of at least four live strains of bacteria, wherein the bacteria comprise Lactobacillus reuteri, Lactobacillus rhamnosus, Lactobacillus Paracasei and Bifidobacterium longum subsp. longum, wherein the at least four live strains of bacteria produce 3-hydroxybutyrate during the fermentation of the probiotic fermented product. 3-Hydroxybutyric acid, also known as 3-hydroxybutyric acid, is an organic compound and a beta hydroxy acid with the chemical formula CH—CH(OH)CH₂CO₂H; its conjugate base is 3-hydroxybutyrate, also known as β-hydroxybutyrate or BHB. β-Hydroxybutyric acid is a chiral compound with two enantiomers: D-β-hydroxybutyric acid and L-β-hydroxybutyric acid. Its oxidized and polymeric derivatives occur widely in nature.

As disclosed herein, the gut epithelial layers are made up of mucin-producing goblet cells and tight junctions (TJ), wherein, the goblet cells and TJs form a paracellular barrier to regulate the flux of ions and solutes along with commensal flora. As further disclosed herein, aging leads to a breakdown of the epithelial barriers of the GI tract, leading to increased infection rates, wherein, loss of gut integrity induces pathological activation of the immune system, and “primes” a proinflammatory environment. Further disclosed herein, the probiotic fermented food is rich in BHB, wherein BHB is the active ingredient in the probiotic fermented food, has anti-inflammatory properties and allows the subject to maintain health against pathogenic bacteria in the gut microbiota, and maintaining the normal balance of gut microbiota helps to improve digestive health as well as the immune system. The BHB in the probiotic fermented food can improve various health conditions such as, for example, dysbiosis, irritable bowel syndrome, diarrhea, high cholesterol, yeast infections and bacterial vaginosis.

In some embodiments, the probiotic fermented food-containing BHB is administered orally to the subject. In some embodiments, BHB producing bacteria are grown in culture, wherein the bacteria are then digested wherein, BHB is then harvested from the digested bacteria, wherein the residue from the bacterial digestion is subjected to phenol chloroform extraction method, wherein the BHB gets dissolved in the chloroform wherein, the chloroform is evaporated and BHB is formulated as a medicament for oral administration. In some embodiments, BHB production can be promoted in a subject as a result of a caloric restriction diet, wherein the calorie restriction diet is a ketogenic or low-carb diet.

### Method of Treating Dysbiosis

Also disclosed herein is a method of treating dysbiosis in a subject. Typical microbial colonies found on or in the body are benign or beneficial. These appropriately sized microbial colonies carry out a series of helpful and necessary functions, such as aiding in digestion. They also help protect the body from infiltration by pathogenic microbes. Dysbiosis is defined by an imbalance in bacterial composition, changes in bacterial metabolic activities, or changes in bacterial distribution within the gut leading to decreased ability to check each other's growth, which can then lead to overgrowth of one or more of the disturbed colonies. Dysbiosis is characterized by GI symptoms, for example, vomiting and diarrhea. Dysbiosis can be a result of aging. In some embodiments, dysbiosis is associated with cancer-treatment, medicine, antibiotics, metabolic syndrome including diabetes, obesity, cardiovascular disorders, kidney and liver disorders, muscle and brain disorders, inflammatory bowel diseases, inflammatory bowel syndrome-dysbiosis. This is characterized by higher levels of inflammatory markers such as IL-6, IL-1β, TNF-α and IL-8 in the blood that have been linked to enhanced senescence.

Chemotherapy accelerates the biological aging processes. Aging-associated dysbiosis can be induced by chemotherapy. Normal aging represents the inevitable, time-dependent decline in physiologic organ function and is characterized by nine hallmarks wherein the hallmarks of aging are genomic instability, telomere attrition, epigenetic alterations, impaired proteostasis, deregulated nutrient sensing, cellular senescence, stem cell exhaustion, altered intracellular communication and mitochondrial dysfunction.

Genomic instability, associated with normal aging, can be caused by physical, chemical, and biological agents. In some embodiments DNA recombination errors or reactive oxygen species can cause genomic instability. Telomere attrition, associated with normal aging, is caused by the decrease in telomerase, an enzyme that augments the lifespan of cells, leading to accumulation of DNA damage at the telomere ends. In some embodiments, cancer treatments for example, azidothymidine and tamoxifen can directly impar telomerase, thereby accelerating aging.

Epigenetic alterations, associated with normal aging, involve changes in DNA methylation, post-translational modifications of histones and chromatin remodeling, all required for normal development. In some embodiments, some cancer treatments for example, doxorubicin, and radiation therapy can lead to DNA hypermethylation and lead to the accelerated aging phenotype.

Impaired proteostasis, associated with normal aging, involves incorrect folding of proteins and improper mechanisms of proteosomal or lysosomal degradation of proteins. In some embodiments, cancer therapeutics, for example ixazomib lead to loss of proteostasis and increased cellular apoptosis. The deregulated nutrient sensing, associated with normal aging, is the reduced function of growth hormones which thereby affect growth and metabolism. In some embodiments, cancer treatments, for example everolimus lead to impaired wound healing, insulin resistance and testicular degeneration, thus promoting the aging phenotype. Cellular senescence, associated with normal aging, is due to cell cycle arrest, wherein its primary purpose is to prevent the propagation of damaged cells. However, senescent cells produce pro-inflammatory cytokines, for example, IL-6, IL-1β, TNF-α and IL-8. Cytokines can contribute to gut dysbiosis. Chemotherapy, CDK4/6 inhibitors, and immunotherapy are aimed at inducing senescence in cancer cells, but they in turn exert a similar cellular senescence in adjacent non-cancerous tissues. Stem cell exhaustion, associated with normal aging, is the decline in the pluripotent function of a stem cell. Altered intracellular communication, associated with normal aging, represents the changes in endocrine, neuroendocrine, and neuronal pathways associated with aging. Immunotherapies, for example, PD-1 blockade can stimulate intercellular communication associated with a proinflammatory phenotype in tum associated with aging. Mitochondrial dysfunction, associated with normal aging, occurs when the efficacy of the respiratory chain is reduced and there is an increase in the accumulation of reactive oxygen species. Chemotherapeutic agents such as cisplatin can generate reactive oxygen species as intermediates which can also cause DNA damage. Any and all of the above-described conditions related to aging can be treated by administering to the subject in need thereof the probiotic composition disclosed herein.

More particularly, the method of treating dysbiosis or aging-associated dysbiosis in a subject can be done by using a probiotic fermented product wherein the fermentation is done by a combination of four live strains of bacteria, Lactobacillus reuteri, Lactobacillus rhamnosus, Lactobacillus Paracasei and Bifidobacterium longum subsp. longum. In some embodiments the probiotic fermented product is administered in combination with a drug, a medicine, exercise, dietary manipulations including weight loss programs, improving gut health, quality of life, nutritional status, or absorption of nutrients in gut from food.

The method of treating dysbiosis or aging-associated dysbiosis in a subject can be accomplished by using a probiotic fermented product wherein the probiotics are in a therapeutically effective dose. In one embodiment, the subject can be a human. As disclosed herein, is a method of ameliorating effects of dysbiosis or aging-associated dysbiosis in a subject, wherein the subject is administered the novel probiotic fermented product at least once a day. In some embodiments, the probiotic fermented milk is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more times until no longer indicated as being needed. In some embodiments, the probiotic fermented milk is administered 1, 2, or 3 times a day until no longer indicated as being needed. In some embodiments, the probiotic fermented milk is administered daily. In some embodiments, the probiotic fermented milk is administered every day, every 2 days, every 3 days, every 4 days, every 5 days, every 6 days, every 7 days, or more. In some embodiments, the probiotic fermented milk is administered every week, every 2 weeks, every 3 weeks, every 4 weeks, or more. In some embodiments, the probiotic fermented milk is administered every month, every 2 months, every 3 months, every 4 months, every 5 months, every 6 months, every 7 months, every 8 months, every 9 months, every 10 months, every 11 months, every 12 months, or more. In some embodiments, the probiotic fermented milk is administered every year, every 2 years, every 3 years, every 4 years, every 5 years, or more. In some embodiments, the

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1

In a first example, is shown the development and optimization of probiotic fermented milk (PFM).

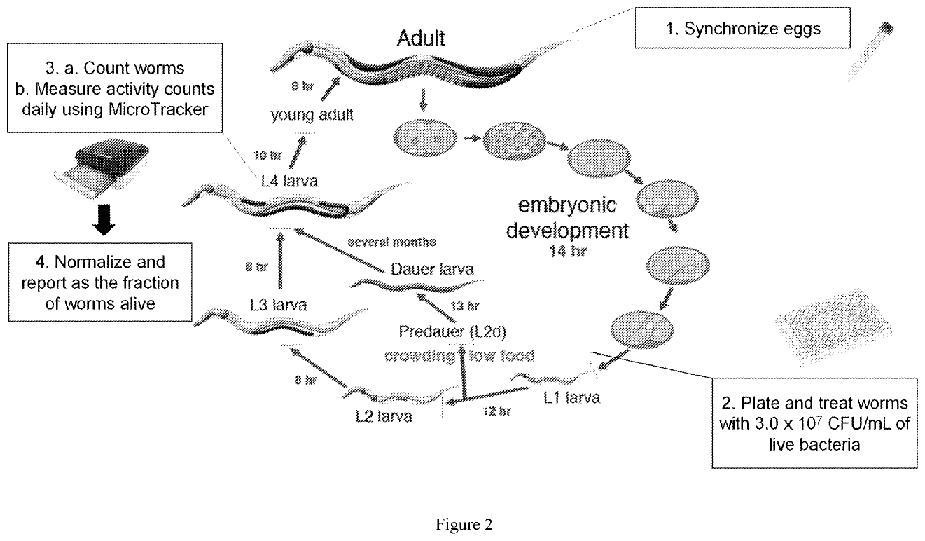

C. elegans embryos develop rapidly and hatch after 14 hours. The first larval stage is completed after another 12 hours, and the animals proceed through four molt cycles before becoming adults. Under crowded conditions or in the absence of food, larvae can choose an alternative developmental pathway leading to the dauer larva, which does not feed but can survive adverse conditions for several months. When life gets better normal development is resumed, the animals exit the dauer larval stage and develop into the normal fourth larval stage before becoming adults. Adult animals are hermaphrodites and produce both sperm and eggs. Over the course of 3-4 days about 300 eggs are laid. The overall life span of C. elegans is 2-3 weeks. The short generation cycle facilitates genetic experiments and is a major advantage for researchers working with this organism (FIG. 2).

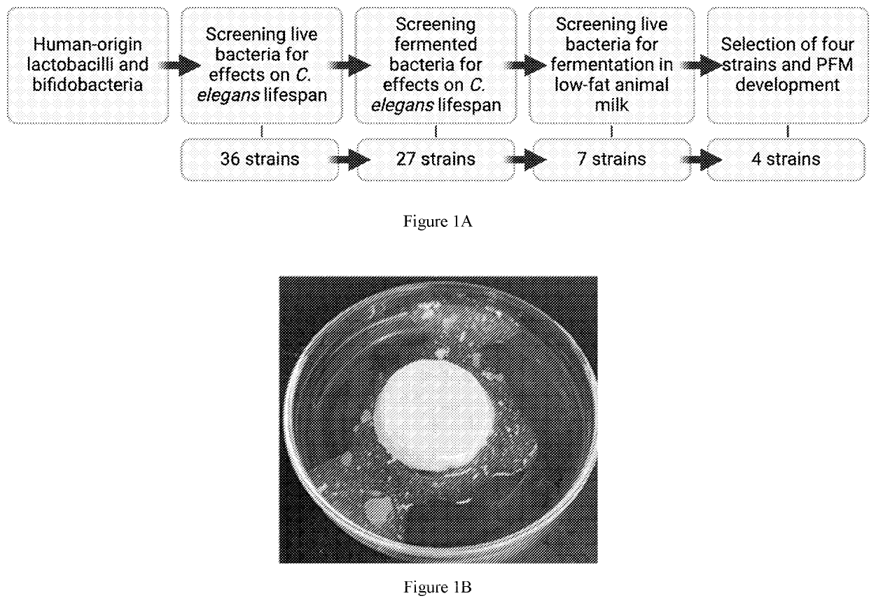

A multi-step screening (FIG. 1A) was performed on 36 human-origin lactobacilli and bifidobacteria that were previously isolated from the infant gut. 36 Human-origin lactobacilli and bifidobacteria were screened live for effects on C. elegans lifespan. Then 27 strains of fermented bacteria were screened for effects on C. elegans lifespan. Seven of those were screened live for fermentation of low-fat animal milk. Out of those seven, four were selected to develop a novel probiotic fermented milk (PFM).

Eggs from adult C. elegans were synchronized, plated, treated with 3.0×10⁷ CFU/ml of live bacteria, normalized, and reported as the fraction of worms alive. Worms were counted and their activity counts were measured daily using MicroTracker.

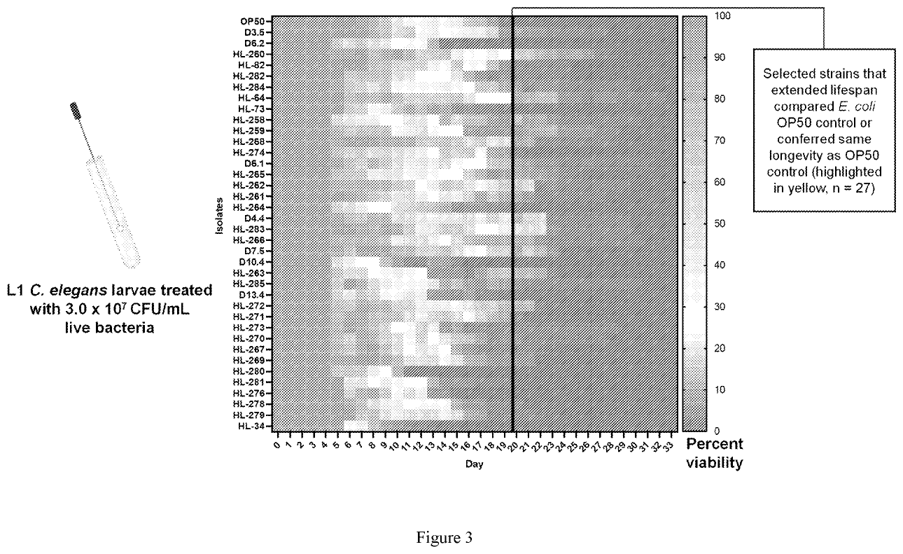

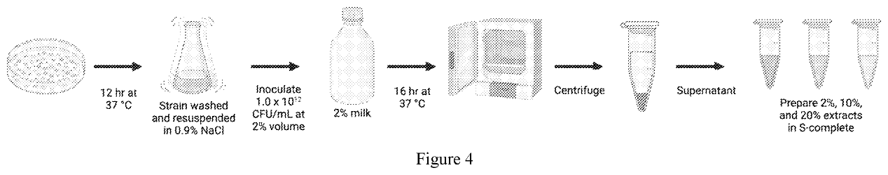

L1 stage C. elegans larvae were treated with 3.0×10⁷ CFU/mL live bacteria. The selected strains were those that extended lifespan compared E. coli OP50 control or conferred same longevity as OP50 control (highlighted in yellow, n=27). Cultures were incubated for 12 hours at 37° C. and then a strain was washed and resuspended in 0.95 NaCl. 1.0×10\hat{ }CFU/ml were inoculated at a 2% volume in 2% milk and incubated at 37° C. for 16 hours, then centrifuged. The supernatant was used to prepare 2%, 10% and 20% extracts in S-complete (FIG. 3).

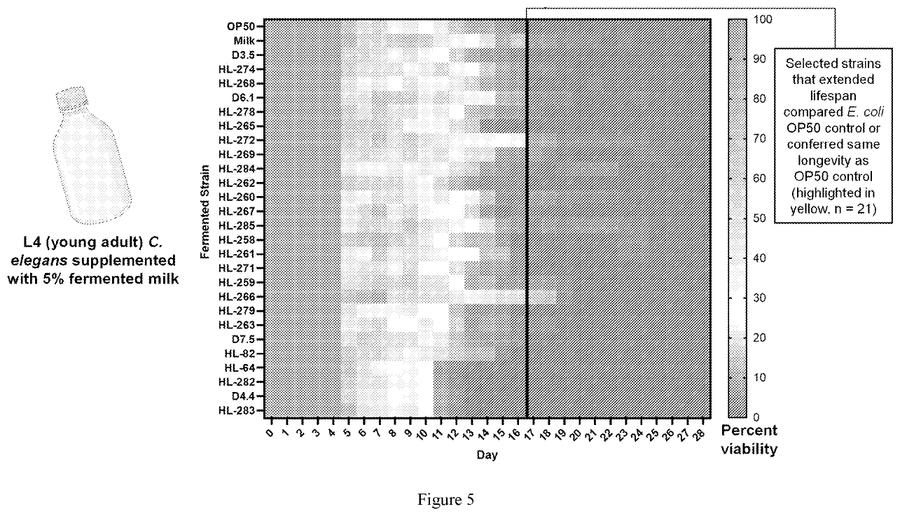

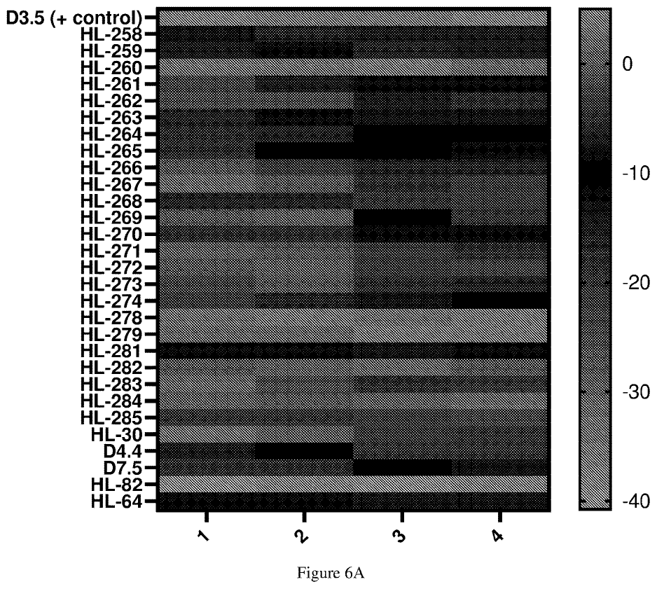

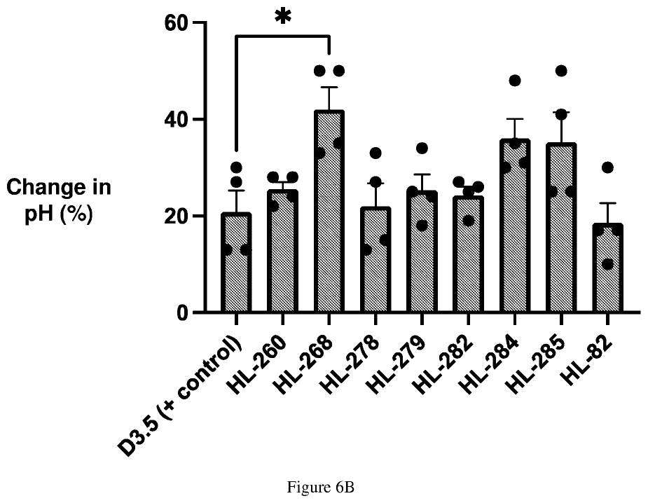

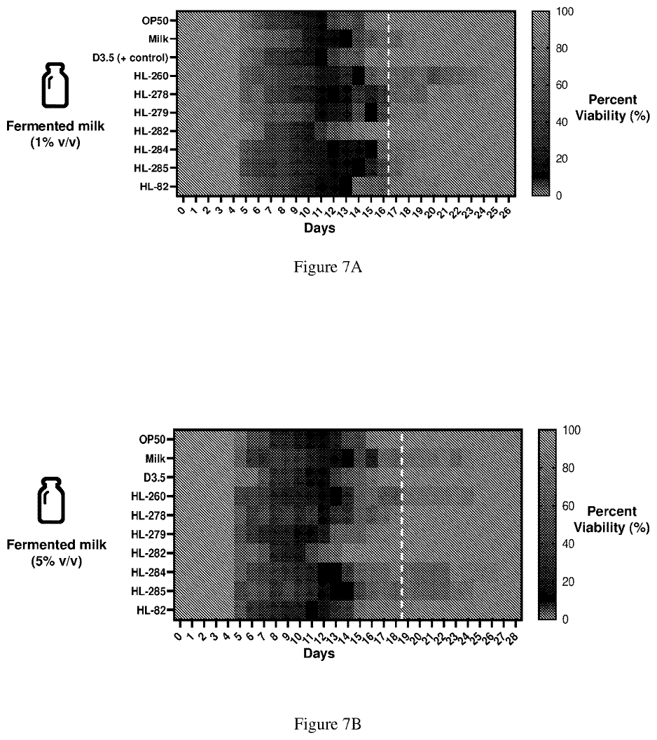

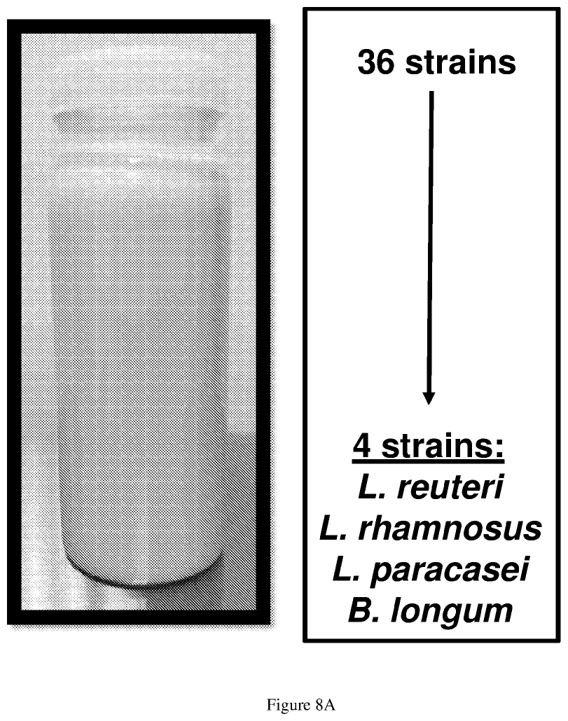

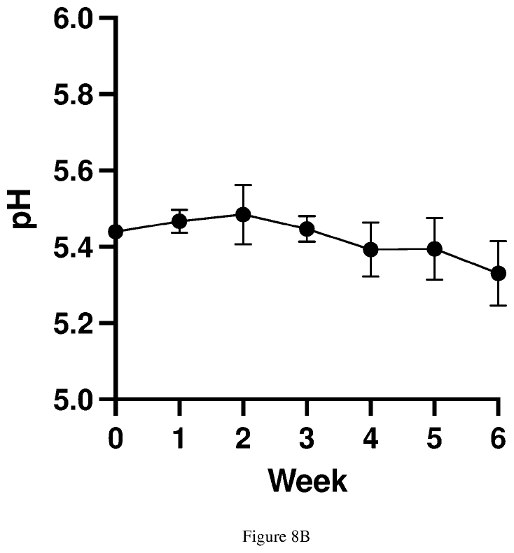

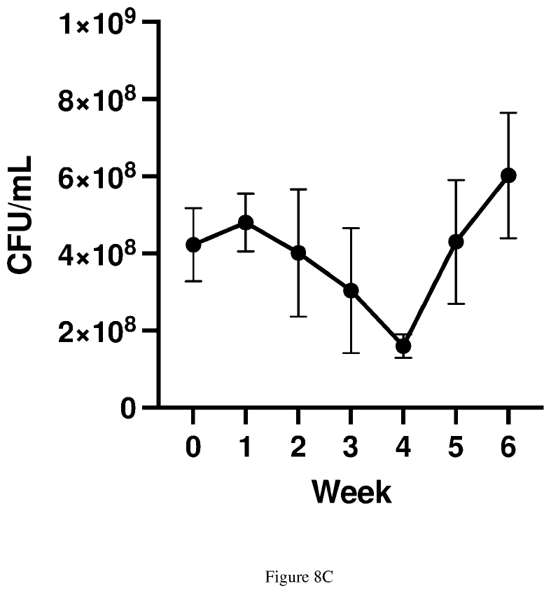

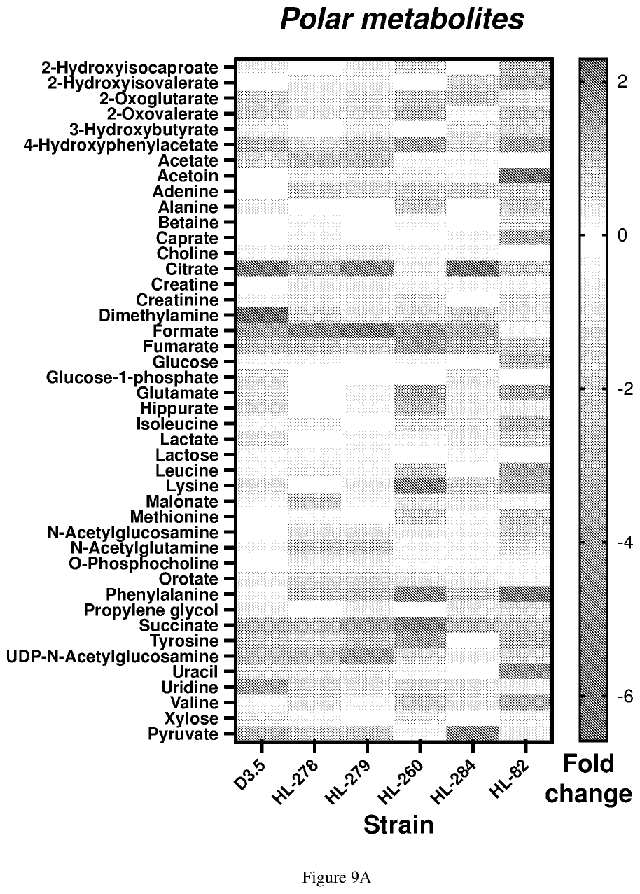

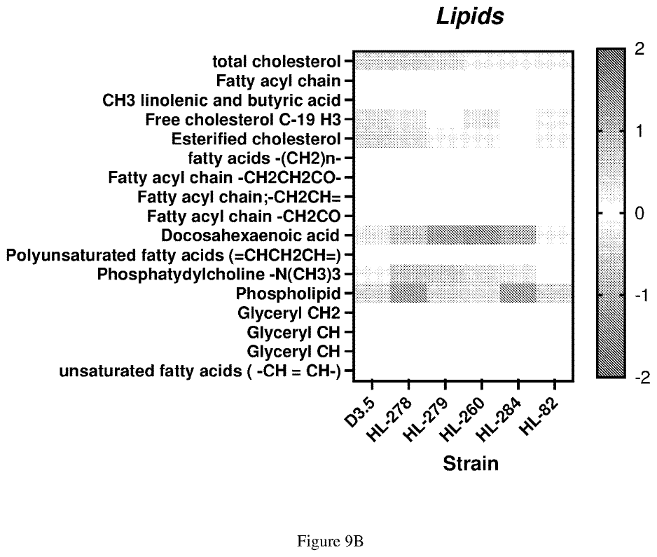

L4 stage (young adult) C. elegans were supplemented with 5% fermented milk. Selected strains were those that extended lifespan compared to E. coli OP50 control or conferred same longevity as OP50 control (highlighted in yellow, n=21) (FIG. 5).

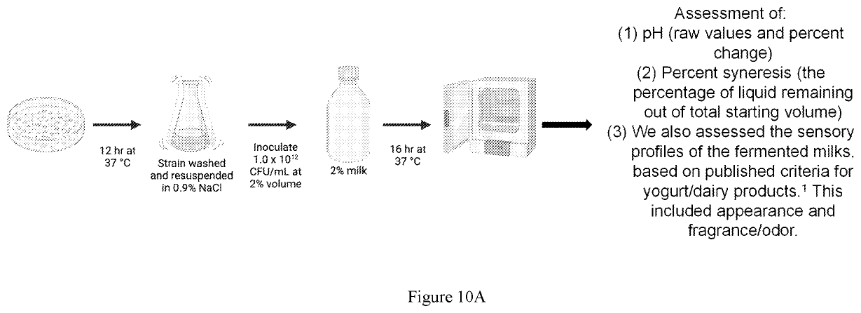

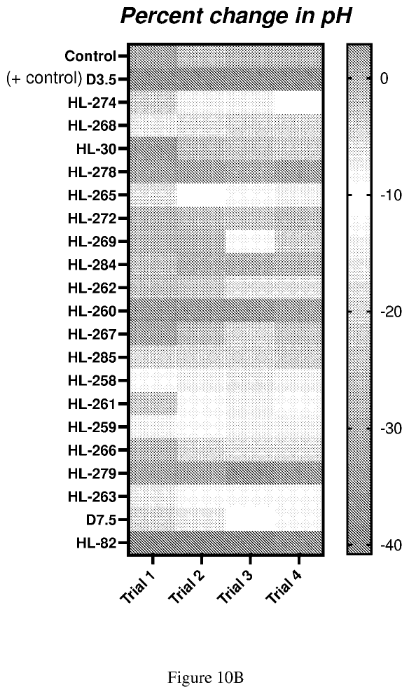

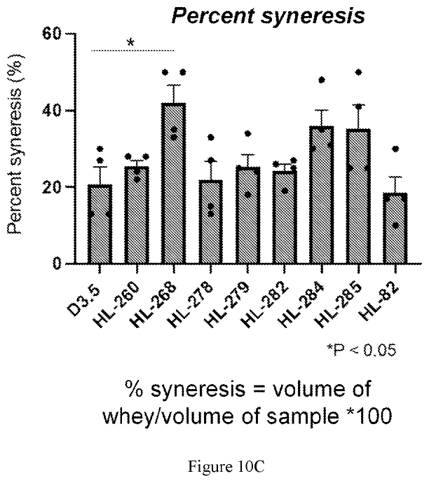

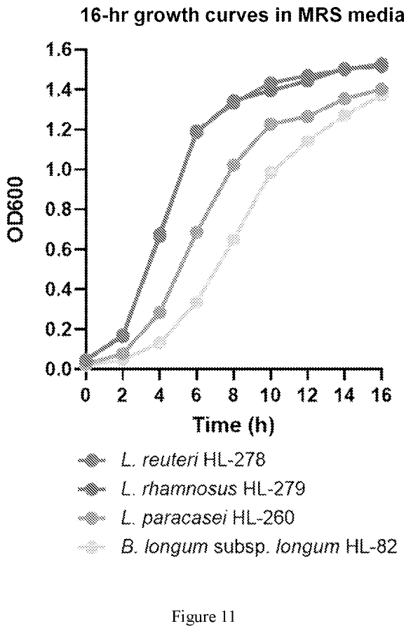

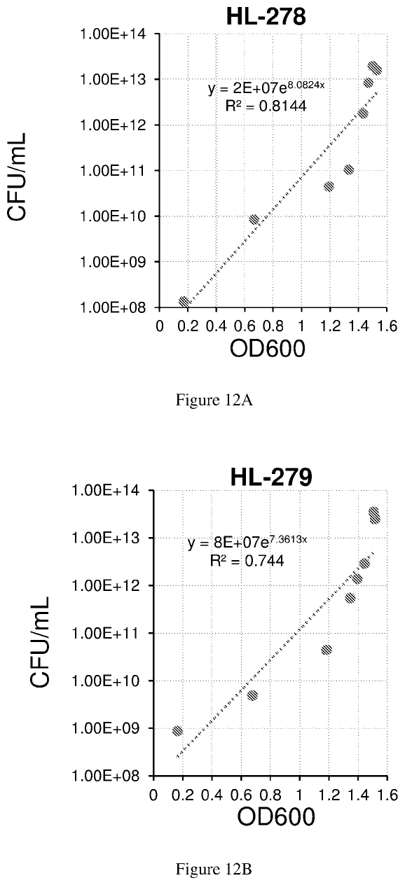

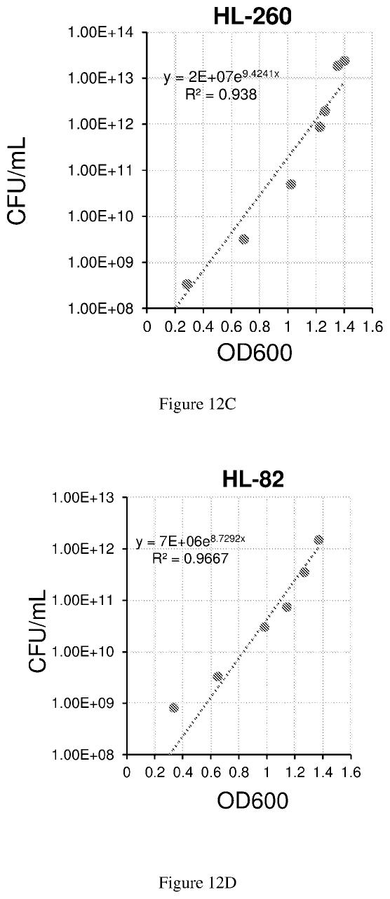

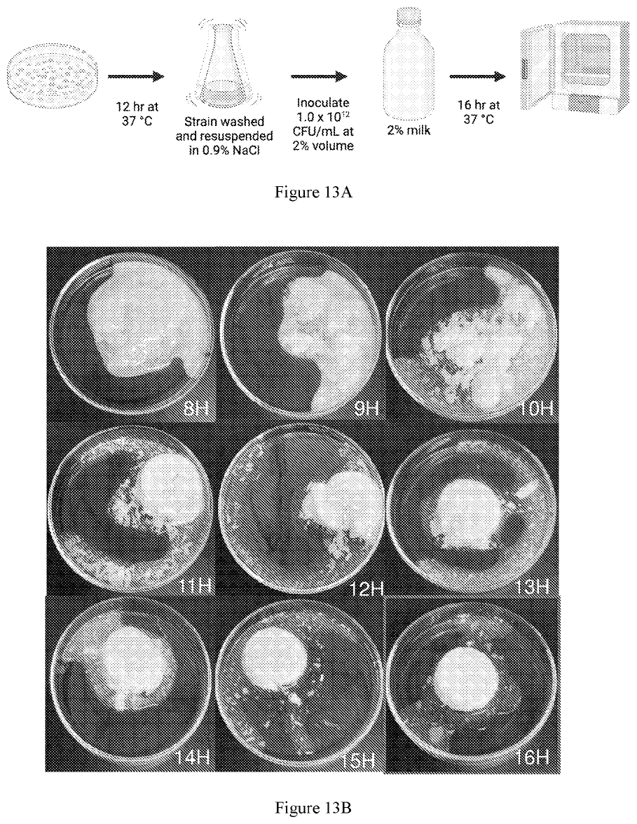

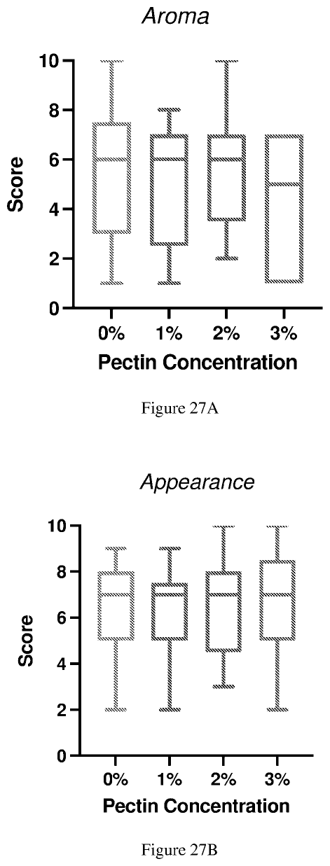

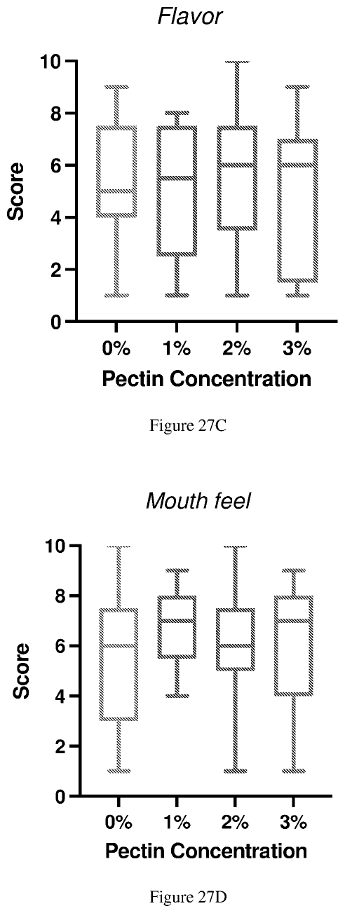

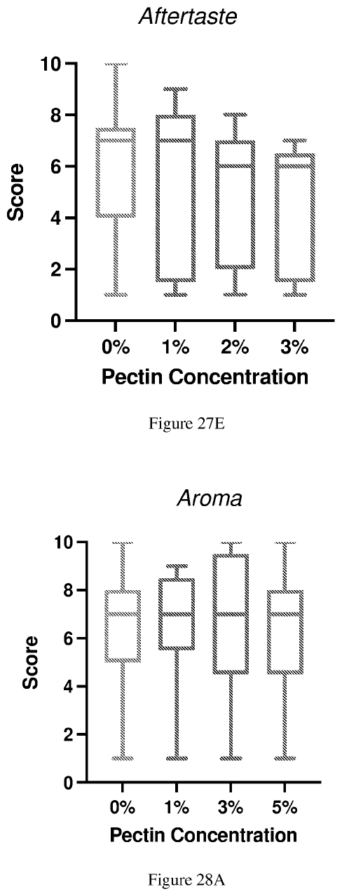

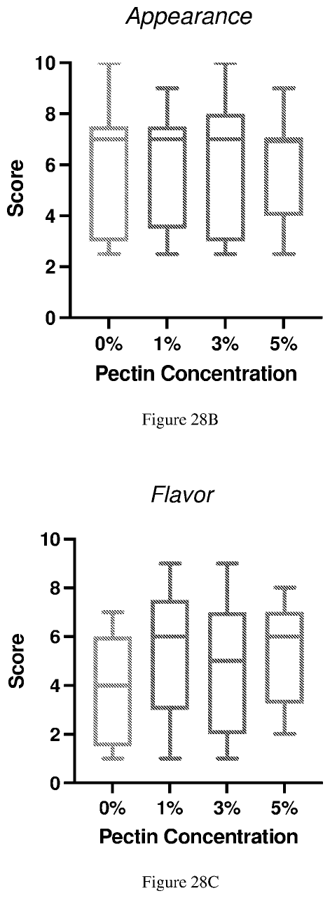

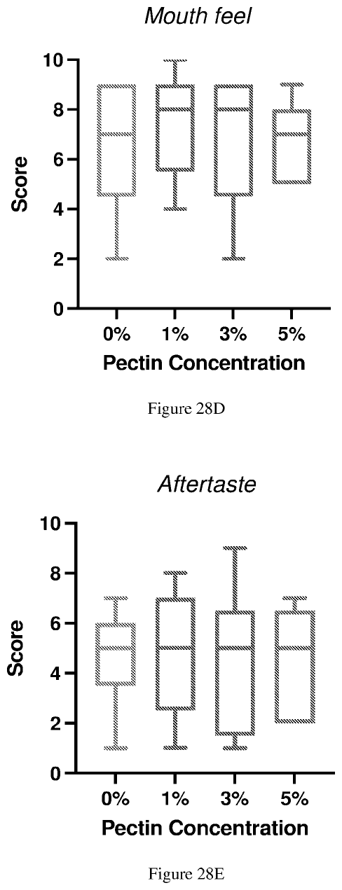

Assessment was performed of:
(1) pH (raw values and percent change) (FIG. 10B).
(2) Percent syneresis (the percentage of liquid remaining out of total starting volume) (FIG. 10C).
(3) The sensory profiles of the fermented milks were also performed based on published criteria for yogurt/dairy products. This included appearance and fragrance/odor. (FIG. 27A-27E).

The selected strains were those that most prominently lowered the pH in low-fat milk and had the lowest percent syneresis. Based on the screenings, four strains were selected that (1) demonstrated anti-aging potential in C. elegans as live bacteria and when fermented in low-fat milk and (2) consistently grew in milk, as assessed by (a) change in pH and (b) percent syneresis Four strains:
1. Lactobacillus reuteri HL-278
2. Lactobacillus rhamnosus HL-279
3. Lactobacillus paracasei HL-260
4. Bifidobacterium Longum Subsp. Longum HL-82

Using these four selected strains, the next step was to optimize their growth in media and milk individually and together, to yield a biologically functional dose of probiotics in our PFM.

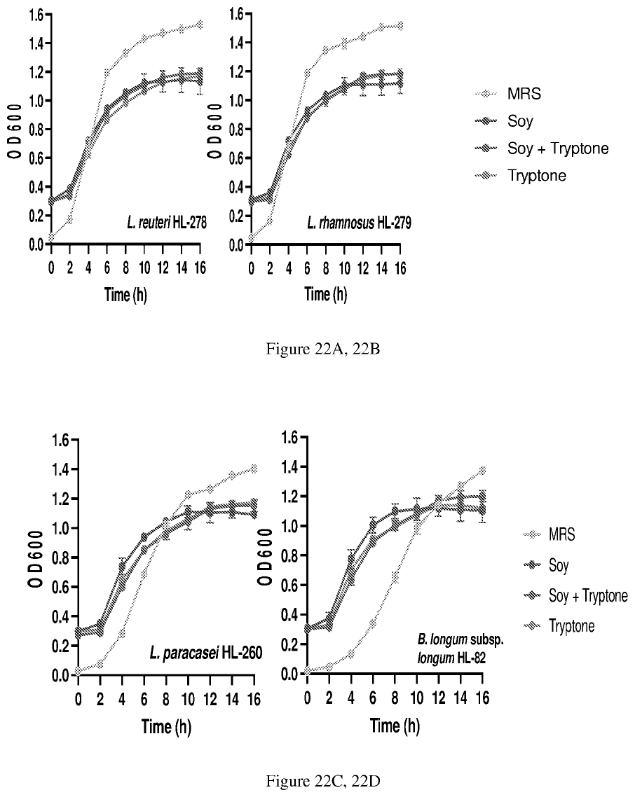

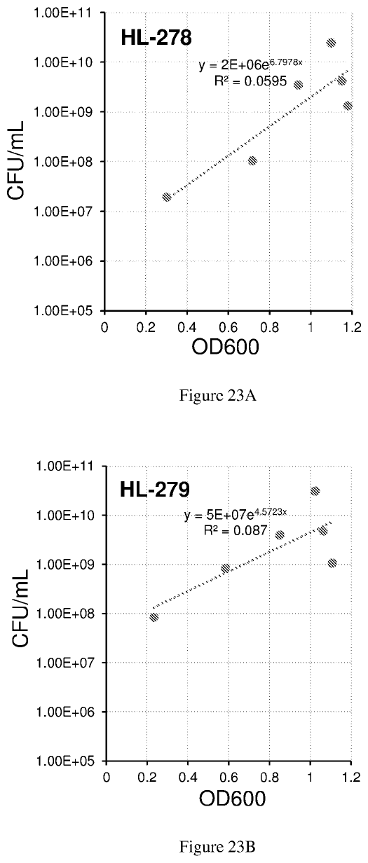

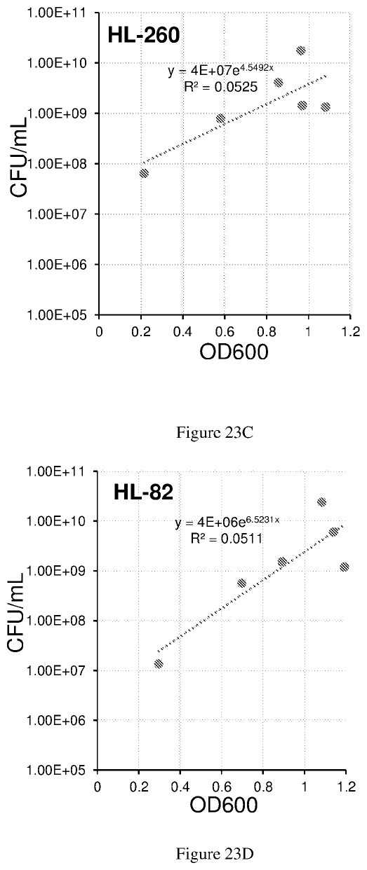

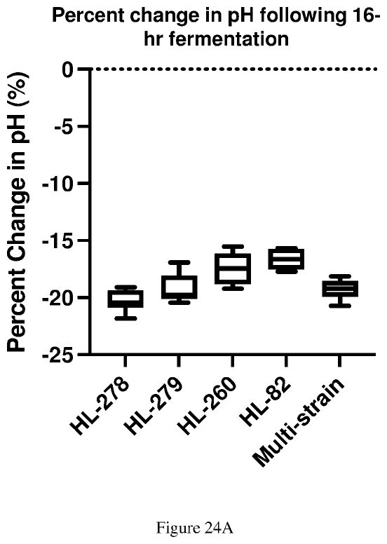

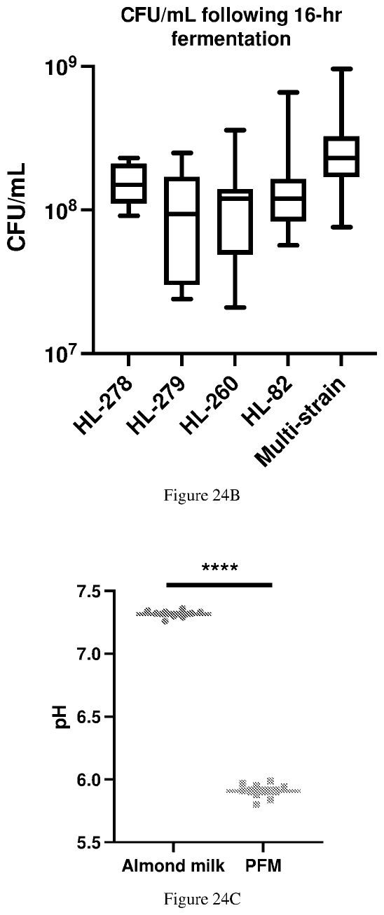

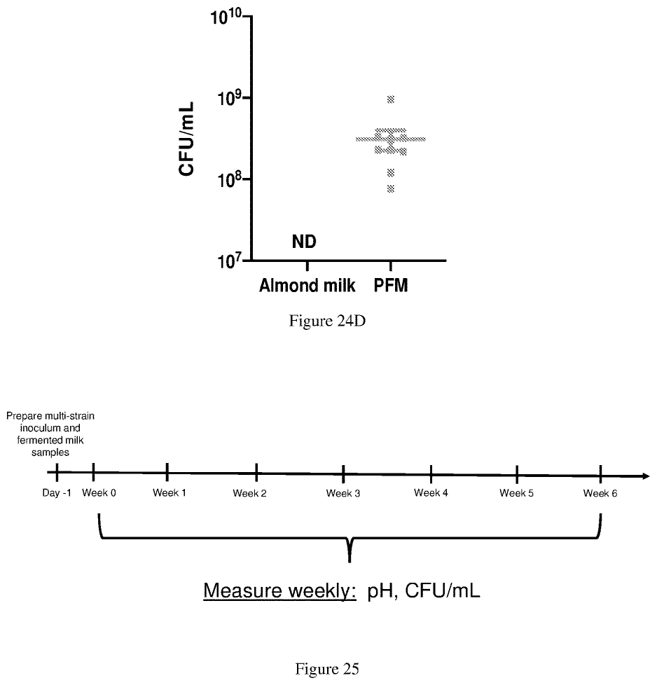

Based on the data obtained for growth curves and standard curves, 12 hours was selected as the optimal growth time for all 4 strains in MRS media (FIG. 22A-22D). For this experiment, the four strains were resuspended in saline together, after being grown in media for their optimized times, and then inoculated into low-fat milk so the total volume of bacteria was 2% of the milk volume.

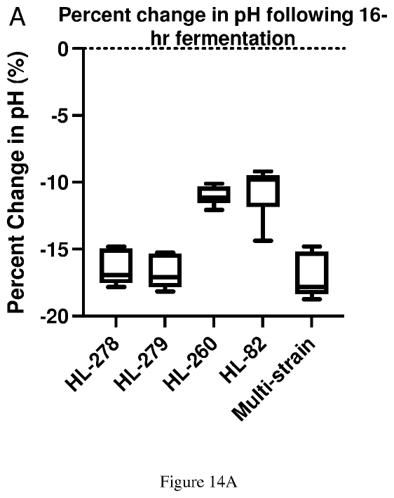

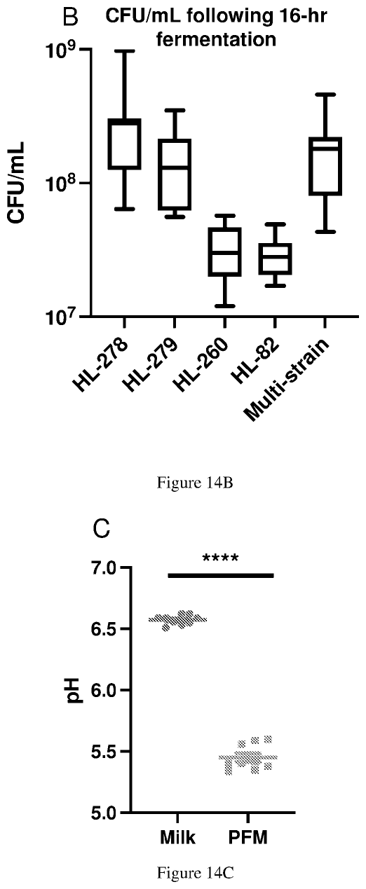

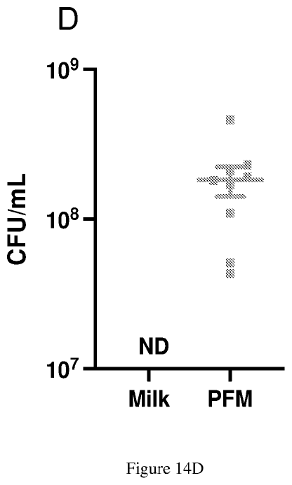

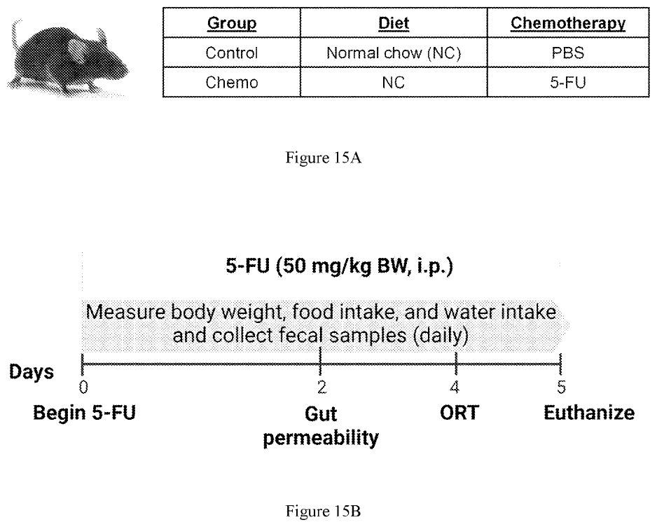

Milk was assessed every hour-starting at 8 hours-by measuring pH and monitoring coagulation. 16 hours was determined to be the optimal fermentation time for PFM. The next step was to confirm growth using pH measures and CFU/mL. Next, it was confirmed that PFM yielded a sufficient number of CFUs (live bacteria) during fermentation. Thus, following the standard protocol, the four strains were inoculated into low-fat milk and assessed pH and live bacteria counts after 16 hours (FIG. 14A-14B). It was found that the multi-strain PFM significantly reduced the pH compared to milk alone and yielded, on average, 108 CFU/mL of milk (FIG. 14C-14D).

### Example 2

In a second example, is shown the determination of the potential protective effects of PFM on cancer treatment-induced dysbiosis in mice.

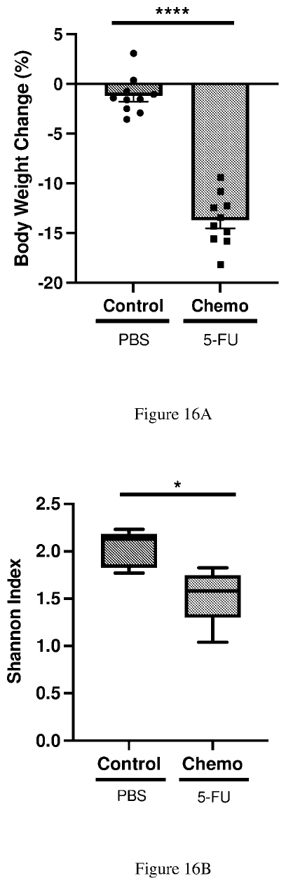

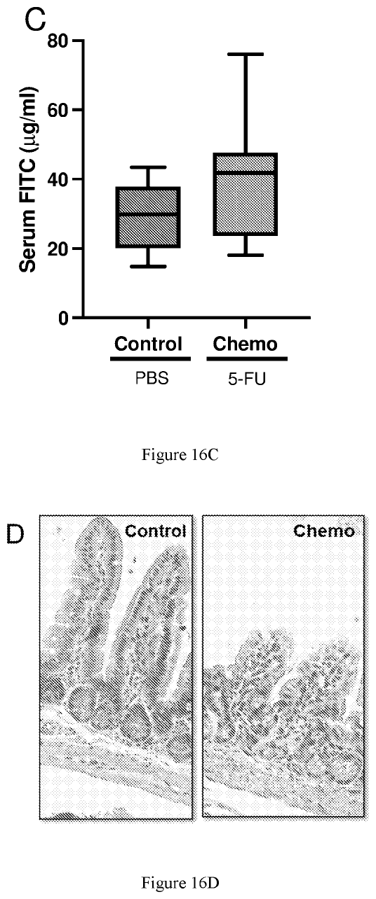

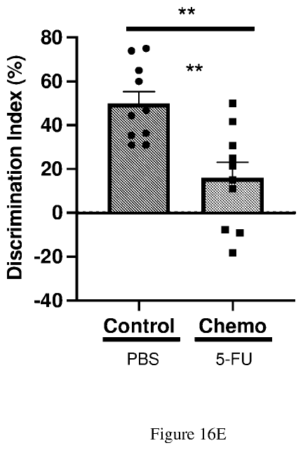

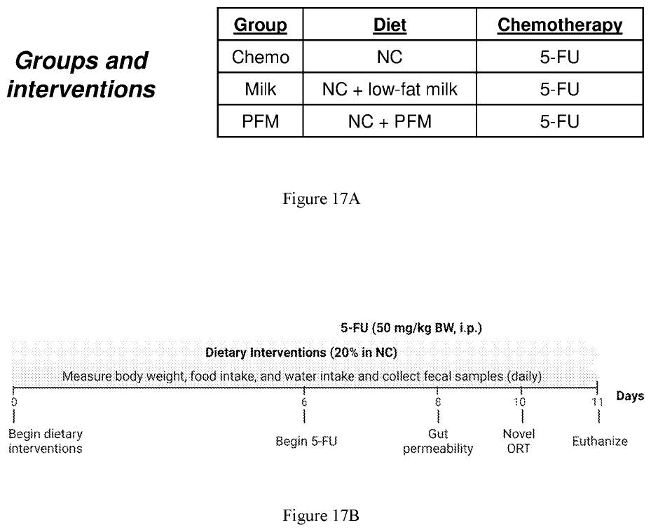

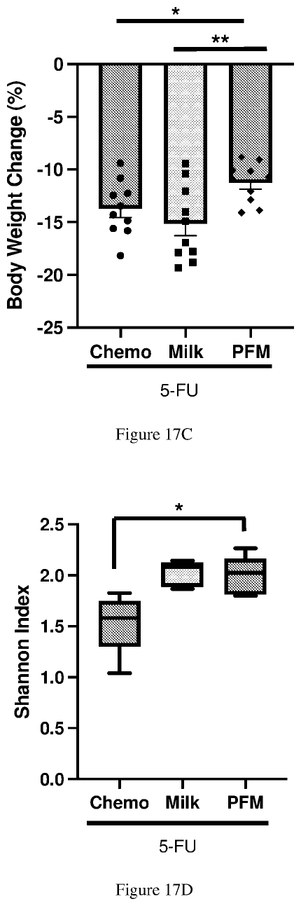

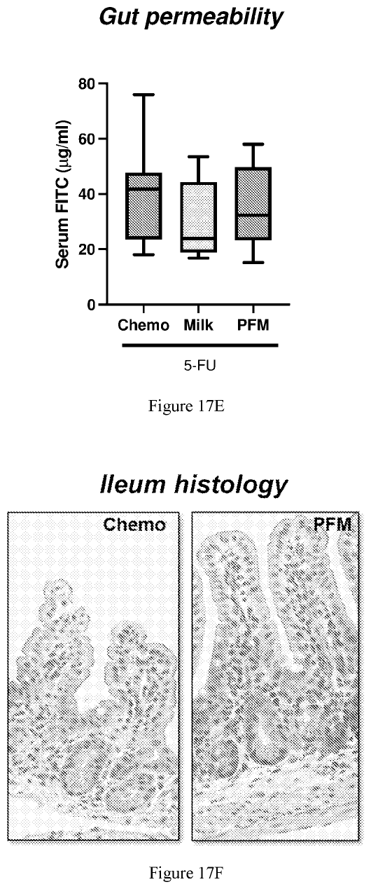

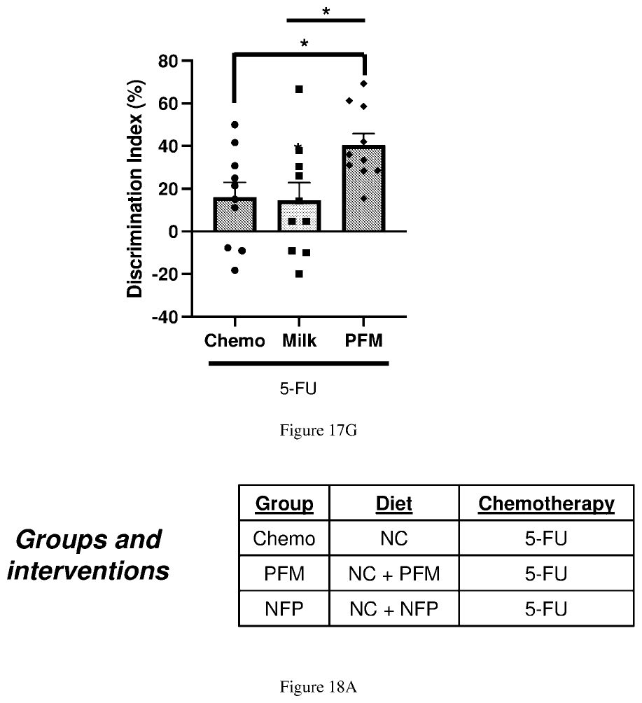

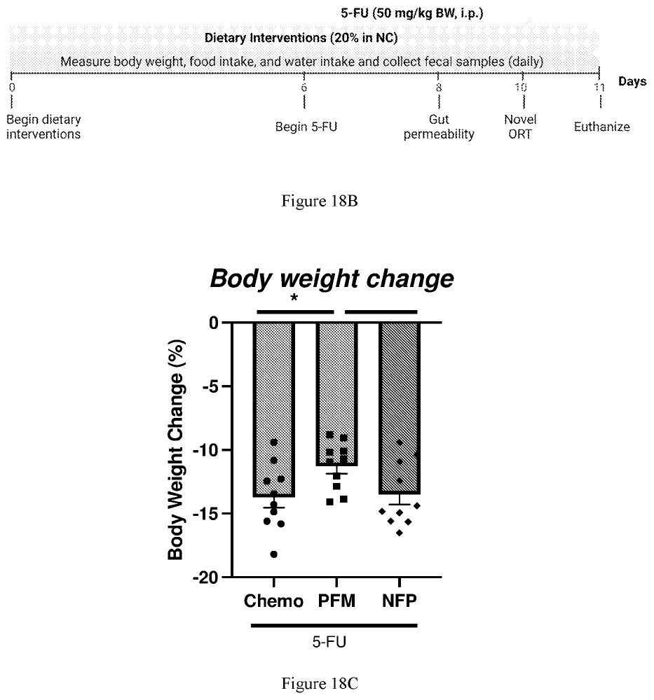

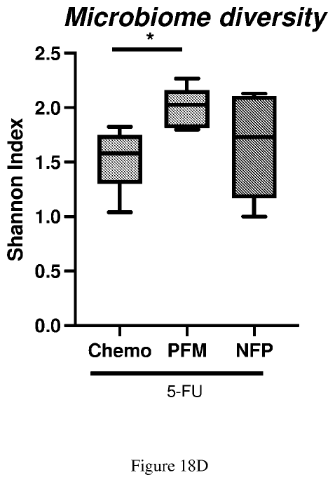

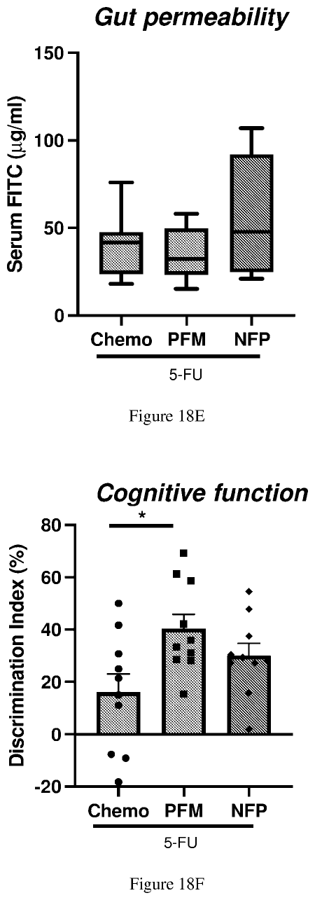

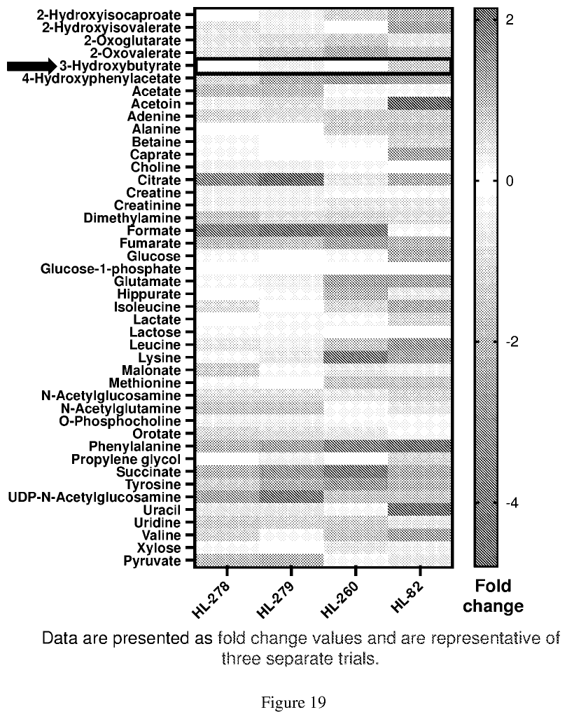

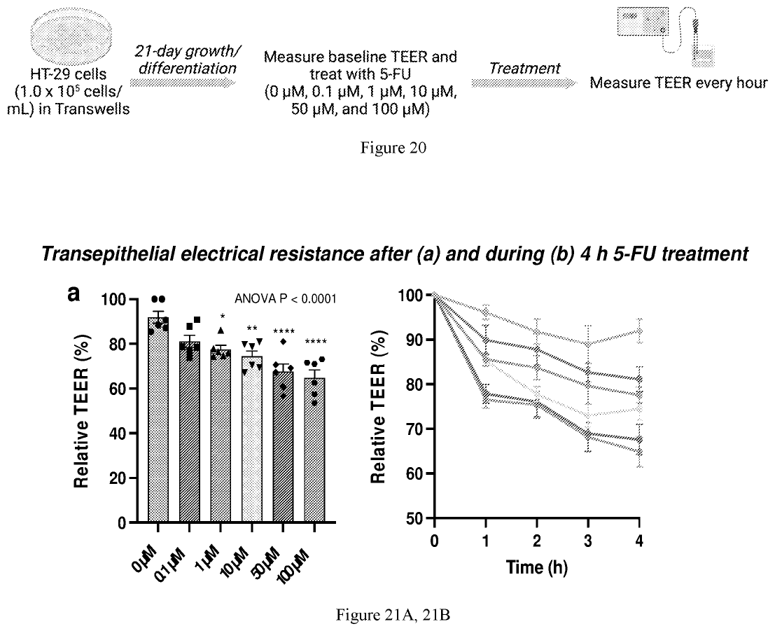

Based on previous studies, a model of chemotherapy induced dysbiosis in C57BL/6 mice using 5-Fluorouracil (5-FU) chemotherapy was established. 5-FU accelerated body weight loss (FIG. 16A), reduced microbiome diversity (FIG. 16B), increased gut permeability (FIG. 16C), damaged intestinal villi in the ileum (FIG. 16D), and impaired cognitive function (FIG. 16E). It was found that PFM significantly protected from the detrimental effects of 5-FU. It was determined where the PFM has more significant protective effects compared to non-fermented probiotics (same four strains suspended in saline, instead of fermented in milk). Interestingly, it was found that PFM significantly protected from 5-FU-induced deficits more so than non-fermented probiotics, leading to questions (1) what ingredient or specific metabolite is contributing to the beneficial effects of PFM? and (2) how is PFM conferring these protective effects? Metabolomics analysis was performed of the individual fermented supernatants of four strains and found that 3-hydroxybutyrate (or β-hydroxybutyrate, BHB) was produced by some of the strains.

### Example 3

In a third example, is shown the determination of the mechanisms underlying the protective effects of PFM.

Using human colon cancer cells (HT-29), an in vitro model was established of 5-FU-induced leaky gut. Transepithelial electrical resistance after (FIG. 21A) and during (FIG. 21B) 4 h 5-FU treatment. Conclusion: 10 μM of 5-FU was selected as a concentration for future experiments.

### Example 4

In a fourth example, is shown optimization of PFM for human consumption for the Microbiome and its modulators in cancer treatment side effects (MiCASE) study.

The four strains were to be developed and optimized in a plant-based medium for more appeal for human consumption. Based on a previous protocol, multiple plant-based MRS medias were produced, using soy peptone, soy+tryptone, and tryptone as the major nitrogen sources. Then the growth of these four bacteria was optimized in (1) plant-based media and (2) almond milk. Conclusion: Tryptone media was used for an optimal growth time of 12-14 hours at 37° C.

Next, to confirm that PFM yielded a sufficient number of CFUs (live bacteria) during fermentation. Four strains were inoculated into almond milk and assessed pH and live bacteria counts after 16 hours. Dextrose was added as a carbon source for the bacteria. It was found that the multi-strain PFM significantly reduced the pH compared to milk alone (FIG. 14A-14C) and yielded, on average, 10⁸ CFU/mL of milk (FIGS. 14B and 14D).

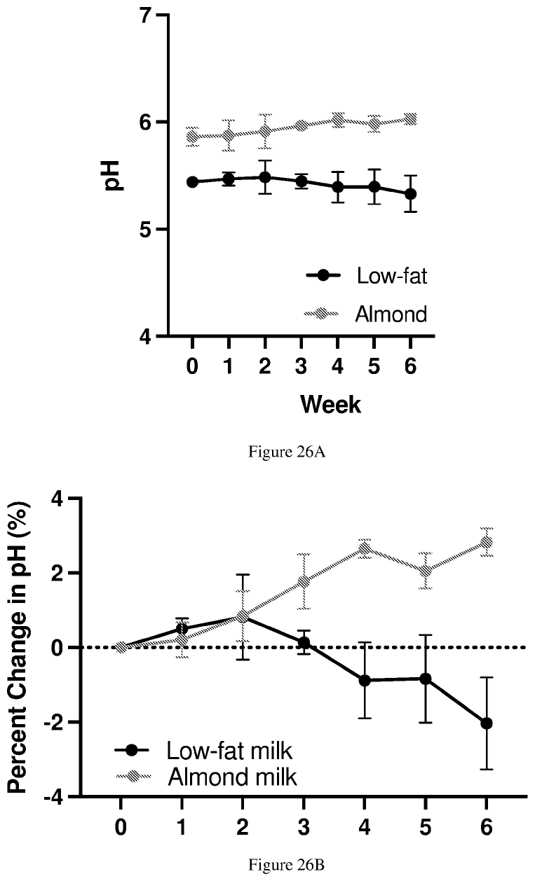

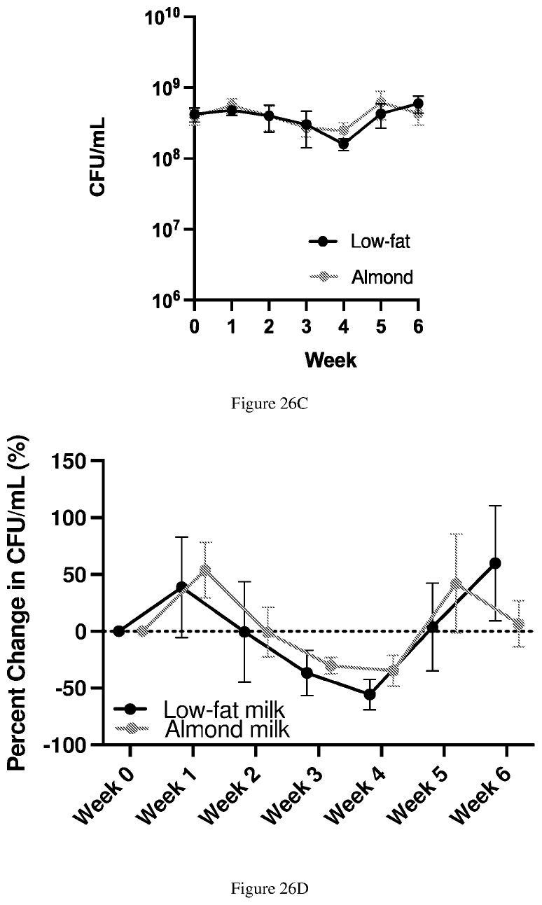

It was next sought to determine the storage stability of PFM (in low-fat and almond milks) at 4° C. pH was measured, and CFU/mL (live bacteria) was quantified weekly (FIG. 26A-26D). Conclusion: The results suggest that our PFMs have 6-week stability at 4° C., based on pH and live bacteria counts.

Sensory evaluations were completed (under USF IRB approval) for PFM by randomly recruiting individuals interested in assessing the formulations based on odor/aroma, appearance, flavor, mouth feel, and aftertaste. During these surveys, the concentration of pectin was optimized (which is commonly used in commercial yogurts as a thickening agent). Conclusion: It was concluded that a 1% pectin concentration was the most favored for both probiotic fermented milk and almond milk.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

1. A probiotic fermented product comprising at least four live strains of bacteria, wherein said bacteria are: Lactobacillus reuteri, Lactobacillus rhamnosus, Lactobacillus paracasei and Bifidobacterium longum subsp. longum.

2. The probiotic fermented product of claim 1, wherein the strain of L reuteri is HL278.

3. The probiotic fermented product of claim 1, wherein the strain of L. rhamnosus is HL 279.

4. The probiotic fermented product of claim 1, wherein the strain of L. paracasei is HL 260.

5. The probiotic fermented product of claim 1, wherein the strain of B. longum subsp. longum is HL 82.

6. The probiotic fermented product of claim 1, wherein the product to be fermented is a milk product.

7. The probiotic fermented product of claim 6, wherein the milk product is low fat milk.

8. The probiotic fermented product of claim 1, wherein the product to be fermented is a vegan or plant-based option.

9. The probiotic fermented product of claim 8, wherein the vegan or plant-based option is selected from but not limited to almond milk, soy milk, oat milk, coconut milk, rice milk or cashew milk.

10. A method of producing 3-hydroxybutyrate, wherein the method comprises:
providing a fermentable food product; and
fermenting the food product by providing a combination of at least four live strains of bacteria, wherein the bacteria comprise Lactobacillus reuteri, Lactobacillus rhamnosus, Lactobacillus Paracasei and Bifidobacterium longum subsp. longum, wherein the at least four live strains of bacteria produce 3-hydroxybutyrate.

11. The method of making 3-hydroxybutyrate of claim 10, wherein the strain of L reuteri is HL278.

12. The method of making 3-hydroxybutyrate of claim 10, wherein the strain of L. rhamnosus is HL 279.

13. The method of making 3-hydroxybutyrate of claim 10, wherein the strain of L. paracasei is HL 260.

14. The method of making 3-hydroxybutyrate of claim 10, wherein the strain of B. longum subsp. longum is HL 82.

15. The method of making 3-hydroxybutyrate in claim 10, wherein the product to be fermented is milk product.

16. The method of making 3-hydroxybutyrate of claim 15, wherein the milk product is low fat milk.

17. The method of making 3-hydroxybutyrate in any of claim 10 wherein the product to be fermented is a vegan or plant-based option.

18. The method of making 3-hydroxybutyrate of claim 17, wherein the vegan or plant-based option is selected from but not limited to almond milk, soy milk or cashew milk.

19. A method of treating dysbiosis in a subject using a probiotic fermented product comprises:
fermenting the food product by providing a combination of at least four live strains of bacteria, wherein the bacteria comprise Lactobacillus reuteri, Lactobacillus rhamnosus, Lactobacillus Paracasei and Bifidobacterium longum subsp. longum, wherein the at least four live strains of bacteria produce 3-hydroxybutyrate. treating the subject with the probiotic fermented product.

20. The method of treating dysbiosis of claim 19, wherein the dysbiosis is aging-associated, cancer-treatment-associated, medicine-associated, antibiotics-associated, metabolic syndrome-, including diabetes, obesity, cardiovascular disorders, kidney and liver disorders, muscle and brain disorders, inflammatory bowel diseases, inflammatory bowel syndrome, associated dysbiosis.

21-33. (canceled)
